(19) United States
(12) Patent Application Publication
(10) Pub. No.: US 2021/0245140 A1
(43) Pub. Date: Aug. 12, 2021

(54) SILICA PROMOTOR FOR PROPANE DEHYDROGENATION CATALYSTS BASED ON PLATINUM AND GALLIUM

(71) Applicant: HALDOR TOPSØE A/S, Kgs. Lyngby (DK)

(72) Inventors: Roy VAN DEN BERG, Copenhagen OE (DK); Rasmus Munksgård NIELSEN, Maaloev (DK); Lived Yegres LEMUS-OLSEN, Copenhagen S (DK); Joachim Harteg JACOBSEN, Birkerød (DK)

(73) Assignee: HALDOR TOPSØE A/S, Kgs. Lyngby (DK)

(21) Appl. No.: 17/049,777

(22) PCT Filed: Mar. 21, 2019
(86) PCT No.: PCT/EP2019/057088

§ 371 Date: Oct. 22, 2020

(30) Foreign Application Priority Data
May 22, 2018 (DK) PA 2018 00227

(51) Int. Cl. B01J 23/62; B01J 23/96; B01J 38/20; B01J 21/12; C07C 5/333

(52) U.S. Cl. CPC B01J 23/62; B01J 23/96; B01J 38/20; C07C 2523/62; C07C 5/3337; C07C 2521/12; B01J 21/12

(57) ABSTRACT

A catalyst for the catalytic dehydrogenation of alkanes to the corresponding alkenes consists of platinum, gallium and optionally potassium on an alumina carrier. Silica has been added to the catalyst, preferably in an amount of 5-10 wt %, as a promotor for the performance thereof.

### TECHNICAL FIELD

The present invention relates to the preparation and use of novel propane dehydrogenation (PDH) catalysts based on platinum and gallium (in the following denoted Pt/Ga propane dehydrogenation catalysts). More specifically, the invention concerns a silica promotor for use in connection with Pt/Ga catalysts for the dehydrogenation of lower alkanes, preferably propane.

### BACKGROUND AND SUMMARY

Basically, the catalytic dehydrogenation of lower alkanes is a simple, but yet important reaction, which can be illustrated by the dehydrogenation of propane to propene in accordance with the reaction:

C₃H₈<->C₃H₆+H₂

With the ever growing demand for light olefins, i.e. lower aliphatic open-chain hydrocarbons having a carbon-carbon double bond, catalytic dehydrogenation is growing in importance. Especially the dehydrogenation of propane and isobutane are important reactions, which are used commercially for the production of propylene and isobutylene, respectively. Propylene is an important fundamental chemical building block for plastics and resins, and the worldwide demand for propylene has been growing steadily for decades. It is expected that the demand growth for propylene will soon be equal to or even higher than that for ethylene. For isobutylene, one of the major applications is that it can be used as feedstock in the manufacture of methyl-tert-butyl ether (MTBE).

The process shown above is endothermic and requires about 125 KJ/mole in heat of reaction. Thus, in order to achieve a reasonable degree of conversion, the dehydrogenation process is taking place at a temperature around 600° C. The dehydrogenation of isobutene is similar to that of propene in every respect, apart from requiring a lower temperature.

Today there are 4 major processes for alkane dehydrogenation in commercial use: The Catofin process, the Oleflex process, the STAR process and the Snamprogetti-Yarzintez process. The differences between these processes primarily deal with the supply of the heat of reaction. The important Catofin process is characterized by the heat of reaction being supplied by pre-heating of the catalyst. The Catofin process is carried out in 3 to 8 fixed-bed adiabatic reactors, using a chromium oxide/alumina catalyst containing around 20 wt % chromium oxide. The catalyst may be supplemented with an inert material having a high heat capacity, or alternatively with a material which will selectively combust or react with the hydrogen formed, the so-called heat generating material (HGM). Promoters such as potassium may be added. During regeneration, coke is burned by contacting the catalyst with an air flow. Simultaneous to the coke combustion, there is usually oxidation of the Cr catalyst, which needs to be reduced again before the dehydrogenation cycle can start again.

Conventional catalyst regeneration processes often do not sufficiently restore the catalytic activity of platinum-gallium based alkane dehydrogenation catalysts to a level equaling that of such catalysts when they are fresh. Thus, skilled persons who practise alkane dehydrogenation, especially PDH, know that decreasing activity of the catalyst inevitably leads to decreasing alkene production, eventually to a point where process economics dictate replacement of the deactivated catalyst with fresh catalyst. Therefore, means and methods to restore catalyst activity more fully are desirable.

To regenerate platinum-gallium based catalysts for alkane dehydrogenation, an oxidation treatment is required. Typically, high temperatures and long reaction times (up to 2 hours) are needed to fully reactivate the catalysts.

Pt/Ga propane dehydrogenation catalysts supported by Al₂O₃ are deactivated very fast during the dehydrogenation procedure. The subsequent regeneration process is not capable of fully recovering the catalyst activity, and therefore a gradual catalyst deactivation is observed from the first regeneration cycle to subsequent regeneration cycles.

It has now surprisingly turned out that the use of a SiO₂/Al₂O₃ combination instead of using Al₂O₃ alone as a catalyst carrier leads to a markedly decreased catalyst deactivation, not only within a single regeneration cycle, but also from the first regeneration cycle to subsequent regeneration cycles. Optimal SiO₂ contents furthermore lead to
increased catalyst activity
improved selectivity and
decreased formation of higher hydrocarbons and coke.

A catalyst based on Pt/Ga also has the advantage of not needing an extra reduction step after regeneration, which is an economic advantage due to the reduction of total cycle time.

Platinum-gallium based catalysts for alkane dehydrogenation are known in the art. Thus, catalysts containing 0.5-2.5 wt % Ga₂O₃, 5-50 ppm Pt, 0.1-1.0 wt % K₂O and 0.08-3 wt % SiO₂ are known from EP 0 637 578 A1, U.S. Pat. No. 5,308,822 A (not containing Pt) and U.S. Pat. No. 7,235,706 A.

In Angew. Chem. Int. Ed. 53, 9251-9256 (2014), a platinum-promoted Ga/Al₂O₃ catalyst is described, which is a highly active, selective and stable propane dehydrogenation catalyst consisting of 1000 ppm Pt, 3 wt % Ga and 0.25 wt % K supported on alumina. A synergy between Ga and Pt is observed, and a bifunctional active phase is proposed, in which coordinately unsaturated Ga³⁺ species are the active species, and where Pt functions as a promoter.

WO 2010/107591 A1 discloses a supported alkane dehydrogenation catalyst with a slightly broader composition range: 0.5-5 wt % Ga or Ga₂O₃, 500 ppm Pt, 0.2 wt % K₂O and 5 wt % SiO₂.

In the above patent documents, the Pt/Ga catalysts are considered to be mostly suited for fluidized bed reactors and not suitable for use in the fixed-bed Catofin process.

WO 2015/094655 A1 describes how to manage sulfur present in a hydrocarbon feed stream while effecting dehydrogenation of hydrocarbons, e.g. propane, present in the feed stream to their corresponding olefins. This is done by using a fluidizable dehydrogenation catalyst that also works as a desulfurant, comprising gallium and platinum on an alumina or alumina-silica support and optionally also an alkali metal such as potassium.

US 2015/0202601 A1 discloses a catalyst and a reactivation process useful for alkane dehydrogenation. The catalyst comprises a group IIIA metal such as gallium, a group VIII noble metal such as platinum, at least one dopant and an optional promotor metal on a support selected from silica, alumina and silica-alumina composites.

A heterogeneous catalyst suitable for alkane dehydrogenation is described in U.S. Pat. No. 9,776,170 B2. It has an active layer that includes alumina and gallia, which is dispersed on a support such as optionally silica-modified alumina.

The present invention presents a solution to the problem of catalyst deactivation during light alkane dehydrogenation, especially Pt/Ga propane dehydrogenation catalysts. So far, Pt/Ga propane dehydrogenation catalysts have not been used commercially for any processes, the main reason for that being that Pt/Ga catalysts simply deactivate too fast. Thus, improving the stability of a Pt/Ga catalyst would allow it to compete with the Cr-based catalysts that are currently used for light alkane dehydrogenation in the Catofin process.

Thus, the present invention concerns a catalyst for the dehydrogenation of alkanes, where lower alkanes are dehydrogenated to the corresponding alkenes according to the reaction

CₙH₂ₙ₊₂<->CₙH₂ₙ+H₂

in which n is an integer from 2 to 5, by feeding the alkane to a catalyst-containing dehydrogenation reactor,

said catalyst consisting of platinum, gallium and optionally potassium on an alumina carrier, wherein silica has been added as a promotor for the performance of the catalyst.

Such catalysts are meant specifically for a fixed-bed process rather than a fluidized bed process.

The catalyst also has the advantage of not needing a reduction step after regeneration (as opposed to the Cr-based catalyst counterpart), which makes the total cycle time shorter.

The catalysts according to the invention preferably contain 0.5-1.5 wt % Ga, 1-100 ppm Pt, 0.05-0.5 wt % K₂O and SiO₂ in an amount of 3-40 wt %, preferably 3-30 wt % and most preferably 5-10 wt %.

The use of SiO₂/Al₂O₃ as a carrier for Pt/Ga catalysts for light alkane dehydrogenation markedly decreases the catalyst deactivation during the dehydrogenation procedure. This improvement allows the catalysts according to the invention to compete with the carcinogenic Cr-based catalysts that are currently used for light alkane dehydrogenation in the Catofin process.

### BRIEF DESCRIPTION OF THE DRAWINGS

FIGS. 1a-1c show the steady-state catalytic performance (5th cycle) regarding activity (FIG. 1a), selectivity (FIG. 1b) and ‘oil’ formation as indicated by the formation of 1-butene (FIG. 1c) of 1.5 g (0.3-0.5 mm) catalyst at a temperature of 570° C., a 12 Nl/h flow of 10% propane and a pressure of 5 bar.

FIG. 2 shows the TPO (temperature-programmed oxidation) of spent catalysts after testing.

### DETAILED DESCRIPTION

The invention is described in further detail in the experimental section which follows.

Experimental

SiO₂ has been identified as a promotor for the performance of Pt/Ga catalysts supported on Al₂O₃. The following procedure was used:

All carriers were impregnated according to the process as described below. Al₂O₃ with different contents of SiO₂ were used as carriers.

Preparation of Impregnation Solution:

4.0 g of a 5 wt % Ga solution, 0.20 g of a 0.5 wt % Pt solution and 0.10 g KNO₃ are dissolved with 11 ml water. This solution is used to impregnate 20 g of the selected support. The sample is rolled for 1 hour to ensure complete pore volume impregnation, dried at 100° C. overnight and then calcined at 700° C. for 2 h with a 4 h heating ramp.

The support materials were the following:
1. Al₂O₃, no SiO₂
2. Al₂O₃, 5 wt % SiO₂, low surface area (SA)
3. Al₂O₃, 5 wt % SiO₂, medium SA
4. Al₂O₃, 5 wt % SiO₂, higher SA
5. Al₂O₃, 10 wt % SiO₂, high SA
6. Al₂O₃, 20 wt % SiO₂, high SA
7. Al₂O₃, 30 wt % SiO₂, high SA

Catalyst Performance:

The reactor used was an isothermal quartz reactor with a quartz thermal pocket over the thermocouple. The outlet gas stream was analyzed using a gas chromatograph with an FID and TCD detector. The gas chromatograph analyzes the C1 to C4 hydrocarbons. Conversion and selectivity are based on the analyzed product mixture. Catalyst performances are evaluated by loading 1.5 gram of catalyst with a sieve fraction of 0.3-0.5 mm into the reactor, and then exposing the catalyst to five cycles of the following sequence of gas flows and temperatures: 200 ml/min of 10% propane in nitrogen for 14 mins at 570° C., followed by 200 ml/min nitrogen flush for 60 mins while heating to 630° C., followed by regeneration with 50 ml/min 2% Oxygen in nitrogen for 30 mins at 630° C., followed by cooling in 50 ml/min 2% Oxygen in nitrogen for 30 mins to 570° C., followed by 200 ml/min nitrogen flush for 3 mins at 570° C. The dehydrogenation cycle is then started again, without including a reduction step. The tests were performed at a pressure of 5 bar.

The results appear from the figures, where:

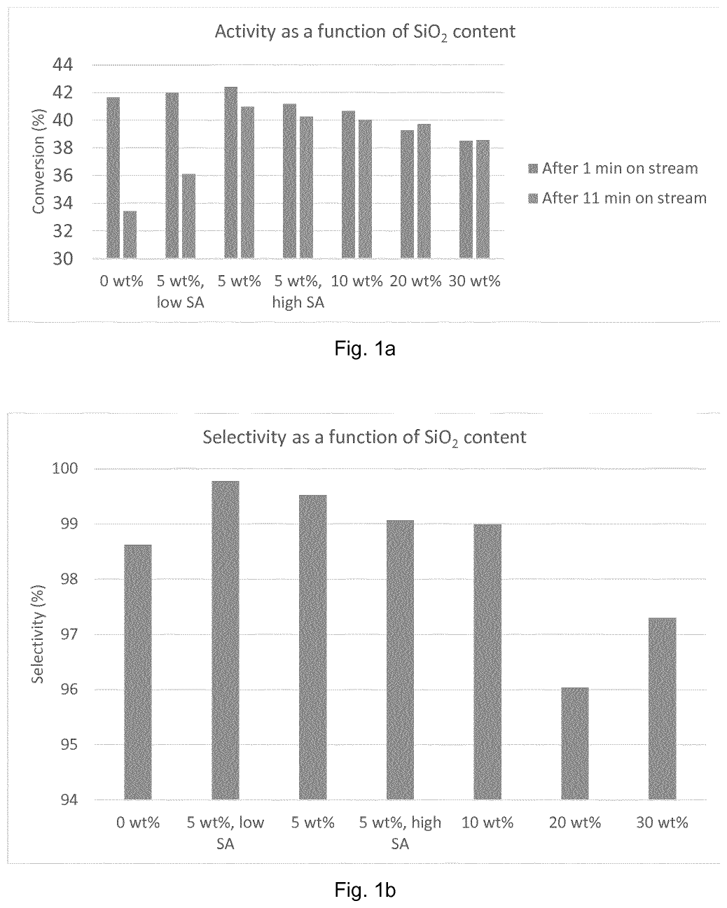

FIGS. 1a-1i show the steady-state catalytic performance (5th cycle) regarding activity (FIG. 1a), selectivity (FIG. 1b) and ‘oil’ formation as indicated by the formation of 1-butene (FIG. 1c) of 1.5 g (0.3-0.5 mm) catalyst at a temperature of 570° C., a 12 Nl/h flow of 10% propane and a pressure of 5 bar, and

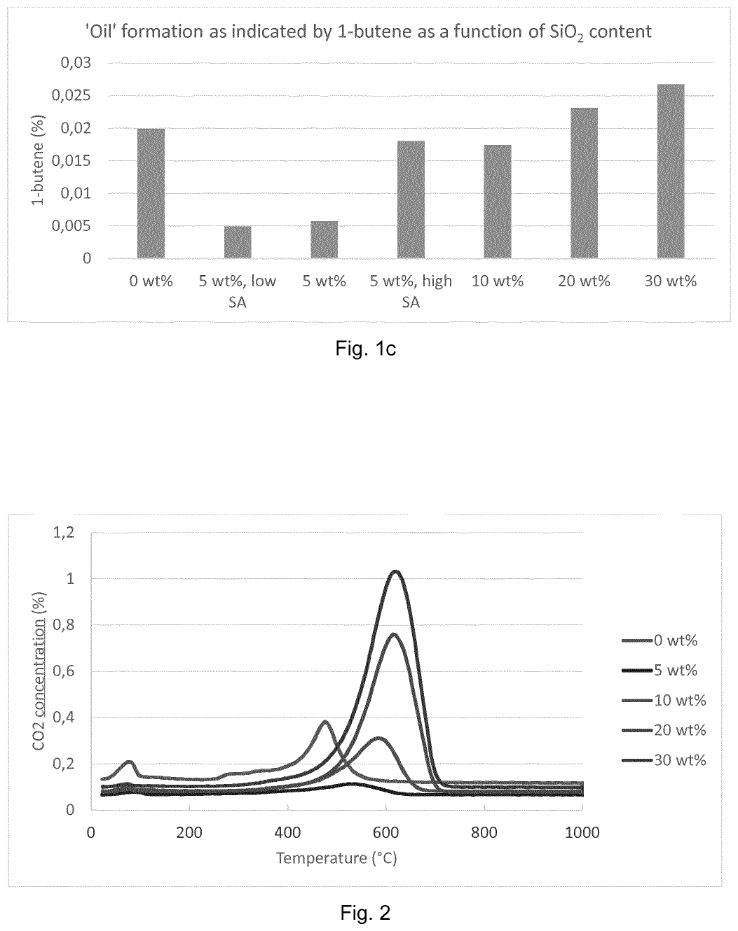

FIG. 2 shows the TPO (temperature-programmed oxidation) of spent catalysts after testing.

It can be seen in FIG. 1a that all SiO₂-containing catalysts have a higher performance after 11 minutes on stream than the corresponding reference catalyst without SiO₂. Two of the catalysts with 5 wt % SiO₂ furthermore also have a higher initial activity after 1 minute on stream. It is thus seen that SiO₂ is able to improve both the activity and the stability of the catalyst.

The catalytic activity of the catalyst seems to correlate very well with the Lewis acidity of the carriers (http://www.sasolgermany.de/fileadmin/doc/aumina/0271.SAS-BR-Inorganics_Siral_Siralox_WEB.pdf). The by-product formation (selectivity), the oil formation and the coke formation all seem to correlate with the Brønsted acidity of the carrier. Furthermore, the higher the SiO₂ loading is, the harder the coke becomes (FIG. 2). It thus requires increasingly higher temperatures to remove the coke. In conclusion, Lewis acid sites introduced by SiO₂ appear to be beneficial for the catalyst, whereas Brønsted acid sites cause side reactions. The optimum catalyst performance seems to be obtained with the 5 wt % SiO₂ carrier.

1. A catalyst for the dehydrogenation of alkanes, where lower alkanes are dehydrogenated to the corresponding alkenes according to the reaction
CₙH₂ₙ₊₂<->CₙH₂ₙ+H₂
in which n is an integer from 2 to 5, by feeding the alkane to a catalyst-containing dehydrogenation reactor,
said catalyst consisting of platinum, gallium and optionally potassium on an alumina carrier, wherein silica has been added as a promotor for the performance of the catalyst.

2. The catalyst according to claim 1, which contains SiO₂ in an amount of 1-40 wt %.

3. The catalyst according to claim 2, wherein the SiO₂ content is 1-30 wt %.

4. The catalyst according to claim 1, which contains 0.5-1.5 wt % Ga, 1-100 ppm Pt and 0.05-0.5 wt % K₂O.

5. A process for the dehydrogenation of alkanes to the corresponding alkenes according to the reaction
CₙH₂ₙ₊₂<->CₙH₂ₙ+H₂
in which n is an integer from 2 to 5 in the presence of a catalyst according to claim 1.

6. The process of claim 5, wherein the catalyst is arranged in a fixed bed.

7. The process of claim 5 comprising periodic cycles of sequential oxidative regeneration steps and said dehydrogenation steps, optionally separated by vacuum or flushing steps, but without a separate reduction step, such as a step in which hydrogen is fed to the catalyst.
